(19) United States
(12) Patent Application Publication
(10) Pub. No.: US 2025/0135456 A1
(43) Pub. Date: May 1, 2025

(54) AUTONOMOUS MICROFLUIDIC DEVICE FOR WATER QUALITY ANALYSIS

(71) Applicant: UNIVERSITY OF PUERTO RICO, San Juan, PR (US)

(72) Inventors: Pedro J. RESTO IRIZARRY, Mayaguez (PR); Katerin RODRIGUEZ PADILLA, Mayaguez (PR); Andrés SAAVEDRA RUIZ, San Juan (PR)

(21) Appl. No.: 18/836,464

(22) PCT Filed: Feb. 7, 2023
(86) PCT No.: PCT/US2023/012505

§ 371 Date: Aug. 7, 2024

Related U.S. Application Data
(60) Provisional application No. 63/307,745, filed on Feb. 8, 2022.

(51) Int. Cl. B01L 3/00; G01N 21/64; G01N 33/18; G16C 20/10

(52) U.S. Cl. CPC B01L 3/502715; G01N 21/6452; G01N 33/18; G16C 20/10; B01L 2200/027; B01L 2200/16; B01L 2300/04; B01L 2300/0861

(57) ABSTRACT

The present disclosure provides autonomous devices, systems incorporating the devices, and methods of using the devices for carrying out assays, such as chemical and/or biological assays. The devices may be microfluidic devices that are capable of carrying out one or more assays concurrently. The devices include one or more reaction wells, one or more channels, an inlet and an outlet. The device may be submerged in a medium containing, for example, a target bacteria. Other embodiments are described and claimed.

### CROSS-REFERENCE TO RELATED APPLICATIONS

This application claims the benefit of and priority to U.S. Patent Application No. 63/307,745, entitled “AUTONOMOUS MICROFLUIDIC DEVICE FOR WATER QUALITY ANALYSIS,” which was filed on Feb. 8, 2022, and which is incorporated herein by reference in its entirety.

### TECHNICAL FIELD

The present disclosure relates to devices and methods for water quality analysis. More particularly, the disclosure relates to autonomous microfluidic devices and methods of using the devices.

### BACKGROUND

Water is the most important natural resource for living beings. It is used in numerous essential daily and recreational activities, such as cooking, bathing, drinking and swimming. The Environmental Protection Agency (EPA) establishes testing procedure guidelines for the analysis of water pollutants under 40 CFR Part 136. Testing procedures include, for example, biological (bacteria, aquatic toxicity), inorganic (metals), organic, pesticides, radiological, and microbiological (bacteria, protozoa) indicators.

The monitoring of total coliforms (E. coli, enterococci, among others) is often used for water quality assessments because it is less costly than testing for a diversified number of microorganisms. Tests developed for bacteria detection should consider sensitivity, specificity, and speed, among other factors.

Conventional methods for fecal bacteria testing in water, such as cell culture, polymerase chain reaction (PCR) and membrane filtration, have high sensitivity and specificity but provide delayed responses. On the other hand, biosensors can detect a pathogenic bacterium using small quantities and can provide information near real-time.

Biosensors are devices capable of transducing biological signals into mechanical or electrical signals capable of being measured, calibrated and displayed for interpretation. Their construction could be sophisticated or relatively simple, and contributions from different scientific fields, such as engineering, biology, physics, chemistry, etc., allow for their use in medicine, the military, and environmental monitoring, for example. One of the most important areas for the application of biosensors is health care due to the need for early, rapid and decentralized diagnosis and maintenance of environmental conditions.

### BRIEF SUMMARY

The present disclosure provides devices, systems, and methods for water quality analysis. In some embodiments, a device comprises a reaction well in communication with an external environment via a channel, the reaction well comprising a matrix, wherein the matrix comprises a reagent to carry out a reaction. The device comprises an impermeable polymer and the channel comprises a first end and a second end, the first end comprising an inlet and the second end comprising an outlet. The reaction well is in fluid communication with the external environment.

In some embodiments, the reaction is a biological reaction, a chemical reaction, a biochemical reaction, or any combination thereof.

In certain embodiments, the reagent comprises a nutrient and optionally an inhibitor. The nutrient may comprise o-nitrophenyl-β-Dgalactopyranoside (ONPG), 4-methylumbelliferyl-β-D-glucuronide (MUG), 4-methylumbelliferyl-β-D-glucoside, or any combination thereof.

In some embodiments, the device comprises a plurality of independent reaction wells and a plurality of independent channels, each well being associated with its own channel.

In some embodiments, the matrix is a solid matrix. In certain embodiments, the matrix comprises an elastomer. In certain embodiments, the matrix comprises polydimethylsiloxane.

In some embodiments, the impermeable polymer is thermoplastic, thermoset, machinable, manufacturable, transparent, translucent, biocompatible, chemically inert, resistant to ultraviolet light, or any combination thereof. The impermeable polymer may comprise substantially no autofluorescence. In certain embodiments, the impermeable polymer comprises methyl methacrylate, polystyrene, polycarbonate, polypropylene, polyethylene, a cycloolefin polymer, and any combination thereof.

In some embodiments, the device comprises a first layer, a second layer disposed on the first layer, and a third layer disposed on the second layer, the second layer defining the channel and the reaction well, and the third layer defining the inlet and the outlet.

In certain embodiments, the inhibitor comprises a salt, a sugar, a phenol, or any combination thereof.

The present disclosure also provides a method of water quality analysis comprising submerging a device in an aqueous medium, wherein the aqueous medium comprises an enzyme, and wherein the device comprises an impermeable polymer. The method also includes transporting the enzyme through a channel to a reaction well in the device, wherein the reaction well comprises a matrix, the matrix comprising a reagent and the reagent is contacted by the enzyme. The method also includes removing the device from the aqueous medium, heating the device, producing a fluorescent signal, and detecting the fluorescent signal.

In some embodiments, the aqueous medium comprises lake water, river water, or ocean water.

In some embodiments, the reagent comprises a nutrient and the enzyme hydrolyzes the nutrient. The nutrient comprises ONPG, MUG, 4-methylumbelliferyl-β-D-glucoside, or any combination thereof. The enzyme comprises β-glucuronidase, β-galactosidase, or a combination thereof.

In certain embodiments, the device is heated for about 60 minutes to about 48 hours. In certain embodiments, the device is heated to a temperature from about 20° C. to about 60° C.

In some embodiments, the impermeable polymer is thermoplastic, thermoset, machinable, manufacturable, transparent, translucent, biocompatible, chemically inert, resistant to ultraviolet light, or any combination thereof. In certain embodiments, the impermeable polymer comprises methyl methacrylate, polystyrene, polycarbonate, polypropylene, polyethylene, a cycloolefin polymer, and any combination thereof.

The present disclosure also provides a method for water quality analysis comprising immersing a device in an aqueous medium, wherein the device comprises a plurality of reaction wells and a transparent, impermeable polymer covering the plurality of reaction wells, wherein each reaction well comprises a matrix having a reagent to carry out a reaction and wherein each reaction well is in fluid communication with the aqueous medium via a channel. The method also includes heating the device at a predetermined temperature after immersing the device; illuminating each of the plurality of reaction wells of the device with a fluorescence excitation source through the impermeable polymer; detecting a fluorescence signal from each of the plurality of reaction wells with a corresponding fluorescence emission sensor through the impermeable polymer, wherein each fluorescence signal is indicative of whether fluorescent activity is detected in the corresponding reaction well; and determining, by a computing device, a water quality metric based on the fluorescence signal from each of the plurality of reaction wells.

In some embodiments, the method further includes sealing the channel for each reaction well after immersing the device. The device is heated after sealing the channel.

In some embodiments, determining the water quality metric includes determining a number of the plurality of reaction wells for which the corresponding fluorescence signal is indicative of detected fluorescent activity; and determining a most probable number (MPN) metric as a function of the number of the reaction wells for which the corresponding fluorescence signal is indicative of detected fluorescent activity. In some embodiments determining the number of the plurality of reaction wells for which the corresponding fluorescence signal is indicative of detected fluorescent activity includes predicting the number with a machine learning model based on the fluorescence signal from each of the plurality of reaction wells. In some embodiments, determining the water quality metric further includes determining a derivative of the fluorescence signal from each of the plurality of reaction well. Predicting the number with the machine learning model further includes predicting the number with the machine learning model based on the fluorescence signal and the derivative of the fluorescence signal from each of the plurality of reaction wells. In some embodiments, the machine learning model is a multi-layer perceptron neural network.

In some embodiments, the method further includes continuing to heat the device, illuminate the plurality of reaction wells, and detect the fluorescence signal from each of the plurality of reaction wells over a first time period between about 30 minutes to about 6 hours; and determining the water quality metric based on the fluorescence signal from each of the plurality of reaction wells after expiration of the first time period.

In some embodiments, the aqueous medium comprises lake water, river water, or ocean water.

In some embodiments, the reagent comprises a nutrient, an enzyme of a bacteria in the aqueous medium hydrolyzes the nutrient to generate a reaction product, and wherein the reaction product generates the fluorescent activity in response to illuminating the plurality of reaction wells. In some embodiments, the nutrient comprises ONPG, MUG, 4-methylumbelliferyl-β-D-glucoside, or any combination thereof. In some embodiments, the enzyme comprises β-glucuronidase, β-galactosidase, or a combination thereof.

In some embodiments, the predetermined temperature is between about 20° C. to about 60° C.

In some embodiments, the impermeable polymer is thermoplastic, thermoset, machinable, manufacturable, biocompatible, chemically inert, resistant to ultraviolet light, or any combination thereof. In some embodiments, the impermeable polymer comprises methyl methacrylate, polystyrene, polycarbonate, polypropylene, polyethylene, a cycloolefin polymer, and any combination thereof.

The present disclosure also provides systems used in water quality assessment. The systems include any of the devices disclosed herein plus a fluorescence excitation source, a fluorescence emission sensor, a heater, a temperature sensor, a microcontroller, and a microcomputer. In some embodiments, the fluorescence excitation source comprises an ultraviolet (UV) light-emitting diode (LED). In some embodiments, the fluorescence emission sensor comprises a camera, a UV sensor, a red/green/blue (RGB) sensor, or any combination thereof. In certain embodiments, the temperature sensor comprises a thermocouple, a thermometer, a thermistor, a resistance temperature detector (RTD) or any combination thereof.

The present disclosure also provides a system for water quality analysis including a device comprising a plurality of reaction wells and a transparent, impermeable polymer covering the plurality of reaction wells, wherein each reaction well comprises a matrix having a reagent to carry out a reaction and wherein each reaction well is in fluid communication with an external environment via a channel. The system also includes a heater coupled to a controller, the controller configured to heat the device at a predetermined temperature after the device is immersed in an aqueous medium. The system also includes a fluorescence excitation source to illuminate each of the plurality of reaction wells of the device through the impermeable polymer; and a fluorescence emission sensor to detect a fluorescence signal from each of the plurality of reaction wells through the impermeable polymer, wherein each fluorescence signal is indicative of whether fluorescent activity is detected in the corresponding reaction well. The system further includes a computing device coupled to the fluorescence emission sensor and configured to determine a water quality metric based on the fluorescence signal from each of the plurality of reaction wells.

In some embodiments, the fluorescence excitation source comprises an ultraviolet (UV) light-emitting diode (LED). In some embodiments, the fluorescence emission sensor comprises a camera, a UV sensor, a red/green/blue (RGB) sensor, or any combination thereof. In some embodiments, the computing device comprises the controller coupled to the heater.

The foregoing has outlined rather broadly the features and technical advantages of the present disclosure in order that the detailed description that follows may be better understood. Additional features and advantages of the disclosure will be described hereinafter that form the subject of the claims of this application. It should be appreciated by those skilled in the art that the conception and the specific embodiments disclosed may be readily utilized as a basis for modifying or designing other embodiments for carrying out the same purposes of the present disclosure. It should also be realized by those skilled in the art that such equivalent embodiments do not depart from the spirit and scope of the disclosure as set forth in the appended claims.

### BRIEF DESCRIPTION OF THE SEVERAL VIEWS OF THE DRAWINGS

A detailed description of the invention is hereafter described with specific reference being made to the drawings in which:

FIG. 1 shows a device according to the present disclosure including a single reaction well;

FIG. 2 shows a three-layered device according to the present disclosure;

FIG. 3 shows a device according to the present disclosure including five reaction wells;

FIG. 4 depicts a system in accordance with some embodiments of the present disclosure;

FIG. 5 shows a side view of certain components of a system in accordance with some embodiments of the present disclosure;

FIG. 6 depicts a system in accordance with some embodiments of the present disclosure; and

FIG. 7 is a simplified flow diagram of at least one method for water quality analysis that may be performed by the system of FIG. 6.

### DETAILED DESCRIPTION

Various embodiments are described below with reference to the drawings in which like elements generally are referred to by like numerals. The relationship and functioning of the various elements of the embodiments may better be understood by reference to the following detailed description. However, embodiments are not limited to those illustrated in the drawings or explicitly described below.

The present disclosure provides autonomous devices and methods of using the devices for carrying out assays, such as chemical and/or biological assays. The devices may be microfluidic devices that are capable of carrying out one or more assays concurrently.

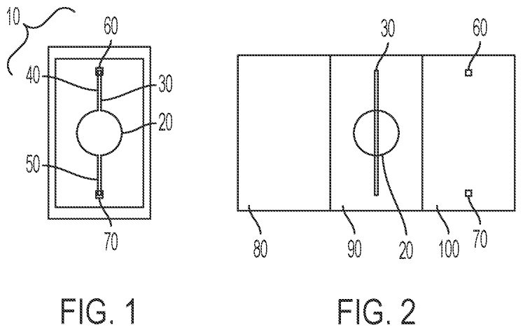

With reference to FIG. 1, some embodiments of the present disclosure provide a microfluidic device 10 that includes one or more reaction wells 20. The reaction well 20 may be disposed within the device 10 and associated with a channel 30. The channel 30 includes a first end 40 and a second end 50. The first end 40 includes an inlet 60 and the second end 50 includes an outlet 70.

The reaction well 20 is disposed within the device 10 such that a top side of the device seals off a top side of the well 20 and a bottom side of the device seals off a bottom side of the well 20. The channel 30 is similarly disposed within the device 10 and can only be accessed by an external environment through the inlet 60 and/or outlet 70.

An external environment may communicate with the well 20 by entering the inlet 60 and traveling through the first end 40 of the channel 30 to the well 20. The external environment may leave the well 20 by traveling through the second end 50 of the channel 30 and out the outlet 70. An external environment may include a gas and/or a liquid, for example.

In some embodiments, if the device 10 is submerged in an aqueous medium, the external environment comprises the aqueous medium. The aqueous medium travels into the inlet 60, through the first end 40, into the well 20, through the second end 50, and out the outlet 70.

The dimensions of the channel 30 are not particularly limited and may be selected by one of skill in the art. In some embodiments, the dimensions are chosen so that, upon submersion in a liquid medium, the channel 30 comprises a hydraulic resistance that is able to be overcome by a hydrostatic pressure of the liquid medium, thereby allowing the liquid of the external environment to enter the inlet 60 and travel to the reaction well 20. The dimensions may be chosen by balancing the hydrostatic pressure under the liquid medium with the hydraulic resistance of the device.

In some embodiments, the channel comprises a depth/height of about 1 μm to about 1,000 μm. For example, the channel may comprise a height of about 1 μm to about 750 μm, about 1 μm to about 500 μm, about 1 μm to about 250 μm, about 1 μm to about 100 μm, about 1 μm to about 50 μm, about 100 μm to about 1,000 μm, about 250 μm to about 1,000 μm, about 500 μm to about 1,000 μm, or about 750 μm to about 1,000 μm.

In some embodiments, the channel comprises a width of about 1 mm to about 10 mm. For example, the channel may comprise a width of about 1 mm to about 9 mm, about 1 mm to about 8 mm, about 1 mm to about 7 mm, about 1 mm to about 6 mm, about 1 mm to about 5 mm, about 1 mm to about 4 mm, about 1 mm to about 3 mm, or about 1 mm to about 2 mm.

In some embodiments, the channel (as measured from the inlet 60 to the outlet 70) comprises a length of about 1 cm to about 10 cm. For example, the channel may comprise a length of about 1 cm to about 9 cm, about 1 cm to about 8 cm, about 1 cm to about 7 cm, about 1 cm to about 6 cm, about 1 cm to about 5 cm, about 1 cm to about 4 cm, about 1 cm to about 3 cm, or about 1 cm to about 2 cm.

The inlet 60 and outlet 70 may comprise any shape and in some embodiments, the inlet 60 may be a different shape than the outlet 70. For example, the inlet 60 and outlet 70 may be independently selected from circular, oval, square, rectangular, and triangular. The inlet 60 and outlet 70 may be of any desired diameter and any desired depth/height.

For example, if the inlet 60 and/or outlet 70 has a circular shape, the diameter may be from about 0.1 mm to about 10 mm, such as from about 0.1 mm to about 8 mm, from about 0.1 mm to about 6 mm, from about 0.1 mm to about 4 mm, from about 0.1 mm to about 2 mm, from about 0.5 mm to about 2 mm, from about 0.5 to about 4 mm, from about 0.5 mm to about 6 mm, from about 0.5 mm to about 8 mm, from about 1 mm to about 3 mm or from about 3 mm to about 5 mm.

If the inlet 60 and/or outlet 70 has a rectangular or square shape, the length and width of each side may be independently selected from about 0.1 mm to about 10 mm, such as from about 0.1 mm to about 8 mm, from about 0.1 mm to about 6 mm, from about 0.1 mm to about 4 mm, from about 0.1 mm to about 2 mm, from about 0.5 mm to about 2 mm, from about 0.5 to about 4 mm, from about 0.5 mm to about 6 mm, from about 0.5 mm to about 8 mm, from about 1 mm to about 3 mm or from about 3 mm to about 5 mm.

In some embodiments, the depth/height of the inlet 60 and the outlet 70 may be independently selected from about 1 μm to about 10,000 μm. For example, the height may be about 10 μm to about 7,500 μm, about 100 μm to about 5,000 μm, about 100 μm to about 2,500 μm, about 100 μm to about 1,000 μm, about 100 μm to about 500 μm, about 1,000 μm to about 10,000 μm, about 3,000 μm to about 10,000 μm, about 5,000 μm to about 10,000 μm, or about 7,500 μm to about 10,000 μm.

Although FIG. 1 depicts an embodiment where the inlet 60 and the outlet 70 are located on the top side of the device 10, the inlet 60 and outlet 70 can be located on a bottom side of the device 10 or on a side of the device 10. The inlet 60 and outlet 70 can be located anywhere on the device so long as they are in communication with the first end 40 and second end 50 of the channel 30. In some embodiments, the inlet 60 and outlet 70 are located on different sides of the device 10.

While FIG. 1 depicts an embodiment where the device 10 comprises a single, unitary body/layer, the device 10 may also be formed from multiple layers and different elements of the device may be disposed in different layers. For example, FIG. 2 shows a base layer 80, a mid-layer 90, and a top layer 100. The mid-layer 90 may be disposed on top of the base layer 80, which may be impenetrable. The mid-layer 90 comprises the reaction well 20 and the channel 30. The top layer 100 is disposed on the mid-layer 90 and includes the inlet 60 and the outlet 70.

The layers may be held together by any manner known in the art, such as by using an adhesive. The adhesive may be, for example, transparent polyester medical-grade double-sided adhesive tape.

While FIG. 1 depicts a device 10 comprising a single layer and FIG. 2 depicts a device 10 comprising three layers, a device 10 of the present disclosure may comprise any number of layers, such as one, two, three, four, five, six or more layers.

Any one or more of the layers may comprise the inlet 60 and outlet 70. Any one or more of the layers may comprise the channel(s) 30 so long as the channel 30 is only exposed to the external environment by the inlet 60 and outlet 70. For example, a channel 30 may be in located in a top side and/or a bottom side of a mid-layer and/or top side of a bottom layer and/or bottom side of the top layer. Also, any one or more of the layers may comprise the reaction well(s) 20 so long as the well 20 is only exposed to the external environment by the inlet 60 and outlet 70 via the channel 30.

Similarly, although the devices 10 of FIGS. 1 and 2 include a single reaction well 20, a device 10 of the present disclosure may include any number of reaction wells, such as from about 1 to about 300, about 1 to about 250, about 1 to about 200, about 1 to about 150, about 1 to about 100, about 1 to about 50, about 1 to about 40, about 1 to about 30, about 1 to about 20, about 1 to about 10, or about 1 to about 5.

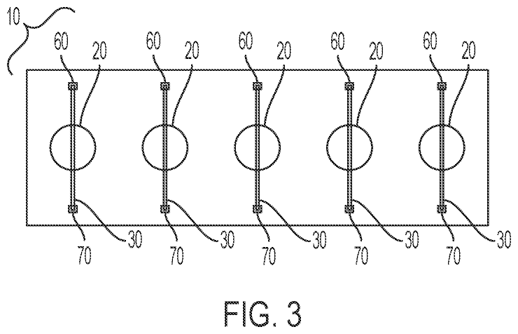

For example, the device 10 of FIG. 3 includes five reaction wells 20. Each well 20 is separate, independent, and sealed off from the other wells such that there is no cross-contamination between wells. Each well 20 has its own channel 30 and, like the devices of FIGS. 1 and 2, each channel is associated with an inlet 60 and an outlet 70.

The reaction well 20 may comprise any shape and in some embodiments, if there are multiple reaction wells, the shape of each well may be independently selected. For example, the shape of a reaction well 20 may be independently selected from circular, oval, square, rectangular, and triangular. The reaction well 20 may be of any desired diameter and any desired depth/height.

For example, if the reaction well 20 has a circular shape, the diameter may be from about 1 mm to about 100 mm, such as from about 1 mm to about 80 mm, from about 1 mm to about 60 mm, from about 1 mm to about 40 mm, from about 1 mm to about 20 mm, from about 1 mm to about 10 mm, from about 10 mm to about 100 mm, from about 30 mm to about 100 mm, from about 50 mm to about 100 mm, or from about 70 mm to about 100 mm.

If the reaction well 20 has a rectangular or square shape, the length and width of each side may be independently selected from about 1 mm to about 100 mm, such as from about 1 mm to about 80 mm, from about 1 mm to about 60 mm, from about 1 mm to about 40 mm, from about 1 mm to about 20 mm, from about 1 mm to about 10 mm, from about 10 mm to about 100 mm, from about 30 mm to about 100 mm, from about 50 mm to about 100 mm, or from about 70 mm to about 100 mm.

In some embodiments, the depth/height of the reaction well 20 may be independently selected from about 1 mm to about 10 mm. For example, the height may be about 1 mm to about 9 mm, about 1 mm to about 8 mm, about 1 mm to about 7 mm, about 1 mm to about 6 mm, about 1 mm to about 5 mm, about 1 mm to about 4 mm, about 1 mm to about 3 mm, or about 1 mm to about 2 mm.

A reaction well 20 in accordance with the present disclosure comprises a matrix (not shown). If the device 10 comprises more than one reaction well 20, each well may include the same matrix or a different matrix. In some embodiments, the matrix is a solid matrix. In certain embodiments, the matrix comprises an elastomer. In certain embodiments, the matrix comprises a hydrogel. In some embodiments, the matrix comprises a silicone-based polymer, such as polydimethylsiloxane (PDMS).

The material(s) and/or polymer(s) used to manufacture the matrix may be moldable, biocompatible, gas permeable, water permeable or slightly water permeable, transparent, UV-resistant, non-fluorescing, and/or hydrophobic.

Moldable properties are beneficial because the polymer/matrix needs to fit within the well 20. Biocompatibility is important because the matrix cannot harm the growing bacteria. Gas and water permeability are beneficial because the matrix covers/encapsulates the reagent until a medium, such as an aqueous medium, fills the well 20 and allows mixing via diffusion/mass transfer across the porous matrix. Transparency is beneficial because the fluorescence from the reagent needs to be seen from outside of the device 10. UV-resistance is important because the device 10 is excited with UV light. The matrix should not fluoresce because if it were to fluoresce, it would interfere with the fluorescent signal of the reagent and the matrix is beneficially hydrophobic because that would protect the reagent and prevent water (humidity) from contaminating the reagent prior to use.

Each well 20 comprises a matrix and each matrix comprises a reagent. The regent may be the same or different in each well 20. In some embodiments, the reagent comprises a nutrient and optionally an inhibitor. If the device 10 comprises multiple wells 20, each well 20 may have the same nutrient or a different nutrient. Further, each well 20 may have the same inhibitor or a different inhibitor (or no inhibitor at all).

Nutrients include methylumbelliferyl derivatives, such as MUG and 4-methylumbelliferyl-β-D-glucoside, in addition to, for example, ONPG. Other nutrients may be selected by one of ordinary skill in the art based on the target bacteria. For example, one of ordinary skill in the art can determine nutrients capable of producing a fluorescent signal when metabolized by an enzyme of the target bacteria.

Nutrients may be selected such that they are specific to the target bacteria. In some embodiments, the nutrient fluoresces when metabolized by an enzyme of the target bacteria. In certain embodiments, the nutrient is hydrolyzed by the enzyme and the reaction product emits a fluorescent signal.

Inhibitors may be present in the reagent if it would be desirable to inhibit growth of non-target bacteria so that only the target bacteria would be able to grow. Inhibitors include, but are not limited to, a salt, a sugar, a phenol, or any combination thereof.

Reagents, nutrients and inhibitors can be selected from those found in, for example, the Colilert™ and Enterolert™ products sold by IDEXX.

In some embodiments, the matrix may include other optional components, such as therapeutic agents, proteins, nucleic acids, peptidenucleic acid conjugates, peptoids, cells, cell extracts, antibiotics, antibodies, viruses, and/or metabolites.

Any manufacturing technique known in the art, such as injection molding, soft lithography, machining, 3D printing, laser ablation, and etching may be used to create device 10. For example, channels and wells may be created using a CO2 laser cutter and the inlet and outlet port may be created using a milling machine.

The device 10 may comprise a polymer and the polymer may be impermeable to solids, liquids, and/or gasses. The polymer may be a thermoset polymer or a thermoplastic polymer, for example. Additionally, the polymer is transparent, semi-transparent, machinable, manufacturable, translucent, biocompatible, chemically inert, resistant to ultraviolet light, and/or any combination thereof. The polymer comprises little or no autofluorescence.

In some embodiments, the polymer comprises methyl methacrylate, polystyrene, polycarbonate, polypropylene, polyethylene, a cycloolefin polymer, or any combination thereof. In some embodiments, the polymer is an acrylic polymer.

In some embodiments of the present disclosure, the entire device, including, for example, all layers of the device, all channels, all wells, etc., are transparent.

The present disclosure also provides systems that comprise the presently disclosed devices. The systems include various components, such as a heater, which may be an incubation heater, a hot plate or a thermoelectric heater, for example. A system may also include a fluorescence excitation source, such as an UV LED. The LED may be specific to the excitation frequency of the reaction product produced by the enzyme/nutrient interaction. Further, a system of the present disclosure may include a fluorescence emission sensor, such as a camera, a CMOS emission detection system, a UV sensor, or an RGB sensor, for image acquisition and data processing. A system may also include a temperature sensor, such as a thermocouple, thermometer, RTD or thermistor. Additionally, a system according to the present disclosure may include a microcontroller, such as the Arduino microcontroller, a microcomputer, such as the Raspberry Pi microcomputer, and the corresponding software to control and use the microcontroller and microcomputer.

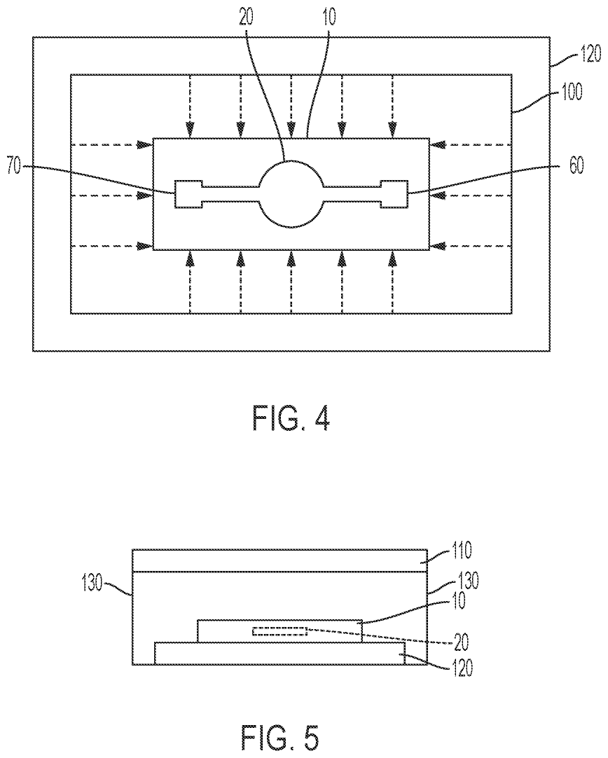

FIG. 4 depicts an embodiment of a system of the present disclosure. Once the medium is introduced into the well 20, the inlet 60 and outlet 70 of the device 10 may be sealed, the device can be placed on a heater 120, and a reaction may occur between the nutrient and an enzyme of the bacteria. A product of that reaction provides a fluorescent signal. The fluorescence excitation source 100 is used to illuminate the device 10 as evenly as possible from all directions. As shown in FIG. 5, a fluorescence emission sensor 110 is placed at an appropriate distance from the well 20. The sensor 110 may be held in place by any means known in the art, such as by using a camera holder or a camera stand. The sensor 110 may comprise walls 130, for example, which block external light. In some embodiments, the walls 130 may provide support for the sensor 110 and keep it suspended over the device 10. In such embodiments, the walls 130 may comprise a material such as metal, plastic, wood, and the like. In certain embodiments, the sensor 110 may be held in place by a stand or some other type of support structure known in the art. If the walls 130 do not need to provide support for the sensor 110, the walls may comprise materials other than metal, plastic, and wood, such as cotton, polyester, or some other type of fabric, for example. The sensor 110 may record images of the well 20 over time so that bacterial growth can be monitored and quantified.

In some embodiments, the microcontroller measures temperature from the heater and a control program to control the flow of current to heater. The microcontroller is connected to the microcomputer and ensures that the desired/required temperature is reached and maintained. Software programs may be generated to enable communication between the various components of the system and execute the testing.

The systems disclosed herein can be used, for example, for water quality analysis. In some embodiments, the systems are used in a method which includes submerging a device as disclosed herein into an aqueous medium, such as fresh water (lake water, river water) or marine water (salt water, sea water, ocean water).

The well(s) of the device comprises the matrix disclosed herein and the matrix comprises a reagent. The reagent may include, for example, a nutrient and optionally an inhibitor.

The aqueous medium may include, for example, a target bacteria, such as Enterococcus faecalis, total coliforms, fecal coliforms, fecal streptococci, Enterococci, and/or Escherichia coli. While the device is submerged, the aqueous medium comprising the bacteria enters the inlet of the device and fills the well and channel. Once the interior of the device is filled with a portion of the aqueous medium, the device may be removed from the aqueous medium and the inlet and outlet may optionally be sealed.

The device may then be placed on a heater while the target bacteria in the aqueous medium interacts with the reagent of the matrix. Heating may continue for about 1 minute to about 48 hours. In some embodiments, the device is heated for about 1 hour to about 48 hours, from about 12 hours to about 48 hours, from about 24 hours to about 48 hours, or from about 36 hours to about 48 hours. An enzyme of the bacteria reacts with the nutrient and produces a reaction product, which emits a fluorescent signal. In some embodiments, the enzyme hydrolyzes the nutrient, thereby producing a reaction product that emits a fluorescent signal.

For example, when 4-methylumbelliferyl-3-D-glucoside is hydrolyzed by an enzyme of the bacteria, it produces 4-methylumbelliferone, which excites at about 365 nm and emits at about 465 nm.

The device may be heated to a temperature of about 20° C. to about 60° C. For example, the device may be heated to a temperature of about 25° C. to about 55° C., about 30° C. to about 50° C., about 35° C. to about 45° C., or about 39° C., about 40° C., about 41° C., about 42° C., or about 43° C.

The enzyme used in the presently disclosed methods may be any enzyme of the target bacteria. For example, E. coli bacteria use β-glucuronidase to metabolize MUG and ONPG. Coliform use β-galactosidase to metabolize MUG and ONPG. β-glucuronidase and β-galactosidase are two illustrative examples of enzymes that can be used in connection with the presently disclosed devices and methods.

In some embodiments, tests conducted using the presently disclosed microfluidic device can be conducted in accordance with one or more of the steps set forth in “Evaluation of Enterolert for Enumeration of Enterococci in Recreational Waters,” G.E. Budnick et al., Applied and Environmental Microbiology, October 1996, Vol. 62, No. 10, p. 3881-3884, the contents of which are incorporated into the present disclosure in their entirety.

The devices disclosed herein are useful in methods for biological and molecular analysis as well as methods for bacterial, pathogen, virus, DNA, molecular, and heavy metal analysis, for example.

The device disclosed herein may be used to carry out a most probable number (MPN) analysis. For example, after incubation/heating the device, the device may be exposed under UV light and the number of fluorescent or “ON” wells may be counted. The number of “ON” wells is related to the MPN of contamination. The unit of contamination is Colony Forming Units (CFU) and is an estimate of the number of viable bacteria in the water sample.

The MPN analysis uses an equation that relates the number of positive wells, the total volume of negative (non-fluorescent) wells, and the total sample volume.

$$MPN = number of positive wells √ ( number of ml in negative wells ) * ( number of ml in all wells ) .$$

If there are no negative wells, the interpretation is that the contamination is higher than the maximum detection limit of the test.

This is merely one example of calculating MPN and one of ordinary skill in the art will understand that additional methods can be used, such as a serial dilution test (https://www.fda.gov/food/laboratory-methods-food/bam-appendix-2-most-probable-number-serial-dilutions).

In certain embodiments, the device may be placed on top of a heater, such as a hotplate, and incubated at about 41° C. for about 24 hours. During this time, the microcomputer activates a fluorescence excitation source, such as a UV light LED array, and the fluorescence emission sensor takes a fluorescent measurement of the well(s). In some embodiments, images are acquired about one time per hour during the 24-hour incubation period and are decomposed into their red, green and blue components. The software in the microcontroller turns on the fluorescence excitation source for a few seconds during each image acquisition. The fluorescent measurement may be analyzed pixel-by-pixel to generate the amount of red, green, blue, and total intensity. These values may be saved in a text file and plotted in an RGB (I) vs. Time plot, where the curves generated are considered as an indicator of the bacteria growth.

In addition to measuring fluorescent signals, the methods of the present disclosure can be carried out by measuring, for example, a luminescent signal and/or a colorimetric signal from the well(s). The size and volume of each reaction well, and the number of reaction wells, are designed to carry out a MPN analysis.

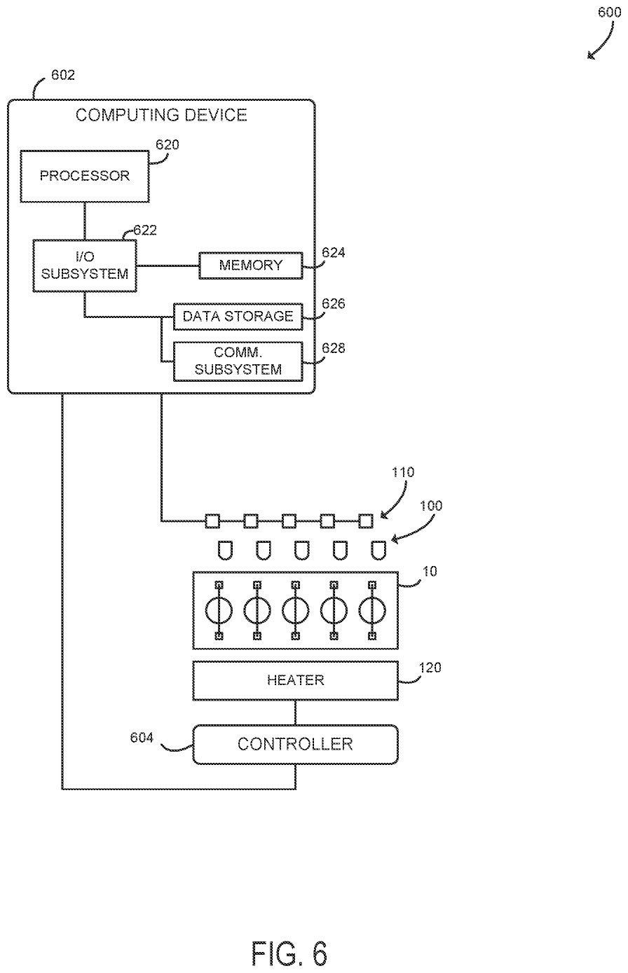

Referring now to FIG. 6, another embodiment of a system 600 of the present disclosure is depicted. As shown, the illustrative system 600 includes a computing device 602 and a controller 604. The controller 604, which may be illustratively embodied as a microcontroller, such as an Arduino microcontroller, a microcomputer, such as a Raspberry Pi microcomputer, or other control device, is coupled to the heater 120 as described above. The system 600 further includes fluorescence excitation source 100, which is illustratively embodied as an array of UV LEDs, and a fluorescence emission sensor 110, which is illustratively embodied as an array of RGB sensors. As shown, a UV LED and an RGB sensor are positioned over each reaction well 20 of the device 10 in order to measure fluorescence in the reaction wells 20. Of course, it should be understood that in other embodiments the system 600 may include a different number and/or arrangement of fluorescence excitation sources and/or emission sensors. For example, the system 600 may include UV LEDs arranged to illuminate multiple reaction wells 20 or sensors (e.g., cameras or other imaging sensors) arranged to capture fluorescence signal data from multiple reaction wells 20.

The computing device 602 may be embodied as any type of device capable of performing the functions described herein. For example, the computing device 602 may be embodied as, without limitation, a microcomputer, a workstation, a desktop computer, a laptop computer, a tablet computer, a smartphone, a server, a rack-mounted server, a blade server, a network appliance, a web appliance, a consumer electronic device, a distributed computing system, a multiprocessor system, and/or any other computing device capable of performing the functions described herein. Additionally, in some embodiments, the computing device 602 may be embodied as a “virtual server” formed from multiple computing devices distributed across a network and operating in a public or private cloud. Accordingly, although the computing device 602 is illustrated in FIG. 1 as embodied as a single computing device, it should be appreciated that the computing device 602 may be embodied as multiple devices cooperating together to facilitate the functionality described below. As shown in FIG. 1, the illustrative computing device 602 includes a processor 620, an I/O subsystem 622, memory 624, a data storage device 626, and a communication subsystem 628. Of course, the computing device 602 may include other or additional components, such as those commonly found in a desktop computer (e.g., various input/output devices), in other embodiments. Additionally, in some embodiments, one or more of the illustrative components may be incorporated in, or otherwise form a portion of, another component. For example, the memory 624, or portions thereof, may be incorporated in the processor 620 in some embodiments.

The processor 620 may be embodied as any type of processor or compute engine capable of performing the functions described herein. For example, the processor may be embodied as a single or multi-core processor(s), digital signal processor, microcontroller, or other processor or processing/controlling circuit. Similarly, the memory 624 may be embodied as any type of volatile or non-volatile memory or data storage capable of performing the functions described herein. In operation, the memory 624 may store various data and software used during operation of the computing device 602 such as operating systems, applications, programs, libraries, and drivers. The memory 624 is communicatively coupled to the processor 620 via the I/O subsystem 622, which may be embodied as circuitry and/or components to facilitate input/output operations with the processor 620, the memory 624, and other components of the computing device 602. For example, the I/O subsystem 622 may be embodied as, or otherwise include, memory controller hubs, input/output control hubs, firmware devices, communication links (i.e., point-to-point links, bus links, wires, cables, light guides, printed circuit board traces, etc.) and/or other components and subsystems to facilitate the input/output operations. In some embodiments, the I/O subsystem 622 may form a portion of a system-on-a-chip (SoC) and be incorporated, along with the processor 620, the memory 624, and other components of the computing device 602, on a single integrated circuit chip.

The data storage device 626 may be embodied as any type of device or devices configured for short-term or long-term storage of data such as, for example, memory devices and circuits, memory cards, hard disk drives, solid-state drives, or other data storage devices. The communication subsystem 628 of the computing device 602 may be embodied as any communication circuit, device, or collection thereof, capable of enabling communications between the computing device 602, the bill pay device 114, the lock box device 116, and/or other remote devices. The communication subsystem 628 may be configured to use any one or more communication technology (e.g., wireless or wired communications) and associated protocols (e.g., Ethernet, Bluetooth®, Bluetooth Low Energy (BLE), Wi-Fi®, WiMAX, 3G LTE, 5G, etc.) to effect such communication.

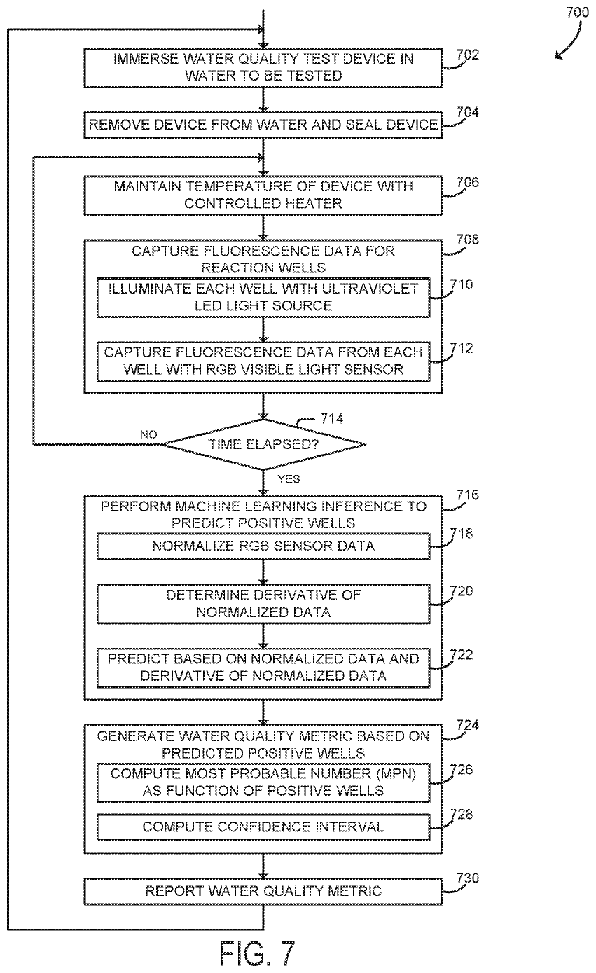

Referring now to FIG. 7, in some embodiments the system 600 may be used to perform a method 700 for water quality analysis. The method 700 begins in block 702, in which a water quality test device 10 is immersed in the water or other aqueous medium to be tested. As described above, when immersed, the reaction wells 20 of the device 10 are self-loaded with the water due to hydrostatic pressure. In block 704, the device 10 is removed from the water and sealed as described above. The device 10 may be placed on the heater 120 or otherwise prepared for testing.

In block 706, the controller 604 maintains the device 10 at a predetermined temperature using the heater 120. For example, the controller 604 may use a thermistor and a Peltier cell to maintain the device 10 at a predetermined temperature, such as 41° C. Of course, other heaters and/or predetermined temperatures may be used in other embodiments. In some embodiments, one or more functions of the controller 604 may be performed by a different device, such as the computing device 602 and/or the heater 120.

In block 708, the computing device 602 captures fluorescence data for each of the reaction wells 20. In block 710, each well 20 is illuminated with the UV LED array 100. In block 712, fluorescence data from each well is captured with the RGB sensor array 110. The fluorescence data may be embodied as digital or analog signals indicative of the intensity of light received by the RGB sensor for each reaction well 20. In particular, the florescence data may include channels or other data indicative of the intensity of each color sensed by the RGB sensor (e.g., R, G, B channels) as well as data indicative of overall intensity or brightness (e.g., intensity (I) or clear (C) channel). The computing device 602 may periodically, continuously, or otherwise capture the fluorescence data over time. For example, the computing device 602 may activate the UV LEDs 100 and the RGB sensors 110 every hour, every minute, or at any other regular or irregular interval.

In block 714, the computing device 602 determines whether sufficient time has elapsed in order to determine a water quality metric. As described above, in some embodiments, the device 10 may be heated for more than 12 hours, more than 24 hours, or between 12 and 48 hours in order to determine a water quality metric. In the illustrative embodiment of FIG. 7, the device 10 may be heated for a shorter duration, such as 6 hours, 3 hours, or a shorter duration. In block 714, the computing device 602 may determine whether a predetermined duration (e.g., 3 hours or 6 hours) has elapsed. If not, the method 700 loops back to block 706 to continue heating the device 10 and measuring the fluorescence signal. If the time has elapsed, the method 700 advances to block 716.

In block 716, the computing device 602 performs a machine learning inference in order to predict a number of reaction wells that are positive for fluorescence activity. As described above, detected fluorescence activity indicates that the target bacteria interacts with the reagent within the reaction well 20. The predicted number of positive reaction wells is determined based on the fluorescence data received from the RBG sensors 110. In block 718, the computing device 602 normalizes the fluorescence data. For example, the computing device 602 may scale all data points to be floating point numbers with zero and one corresponding to the minimum and maximum values reported by the RGB sensors, respectively. In block 720, the computing device 602 determines a derivative of the normalized data. In block 722, the computing device 602 predicts the number of positive wells based on the normalized data and the derivative of the normalized data. In particular, the computing device 602 may supply the normalized data and the derivative data as inputs to a trained machine learning model such a multi-layer perceptron neural network (MLPNN). The machine learning model may classify each reaction well as positive or not positive based on those inputs.

In an illustrative embodiment, the MLPNN includes four input neurons, which is the same size as the input data (R, G, B, I). In some embodiments, the MLPNN further includes about two hidden layers, about four hidden layers, about six hidden layers, or more, and each hidden layer includes a number of neurons. For example, the MLPNN may further include two hidden layers of eight and 16 neurons, respectively, or the MLPNN may further include four hidden layers of 32 neurons per hidden layer. The activation function for the first two stages is ReLU, and the activation function for the last stage is a sigmoid activation function. The MLPNN may be trained using experimental data captured for a device 10 using known bacteria loading. The learning rate is 0.0005. Although illustrated as receiving both the normalized RGBI data and the derivative data as input, it should be understood that in some embodiments, the MLPNN may be used with just the normalized RGBI data as input or with just the derivative data as input. However, prediction accuracy may be improved by using both normalized RBGI data and derivative data as input to the MLPNN. Further, although illustrated and described as using an MLPNN, it should be understood that other machine learning models may be used in other embodiments, such as support vector machines, random forests, multiple linear regression, or other machine learning models.

In block 724, the computing device 602 generates a water quality metric based on the number of predicted positive wells. In block 726, the computing device 602 computes a most probable number (MPN) as a function of the number of wells. In block 728, the computing device 602 may compute a confidence interval.

In block 730, the computing device 602 reports the water quality metric. The computing device 602 may display, store, transmit, or otherwise provide the water quality metric to a user and/or to another system for further analysis or processing. By using a trained machine learning model, the computing device 602 is capable of predicting positive wells with sufficient accuracy after a shorter incubation period compared to other techniques. For example, MPN analysis may be performed in 3 hours or 6 hours, which is an improvement over other testing techniques. After reporting the water quality metric, the method 700 may loop back to block 702, in which additional water samples may be tested.

The foregoing may be better understood by reference to the following examples, which are intended for illustrative purposes and are not intended to limit the scope of the disclosure or its application in any way.

### EXAMPLES

In one non-limiting test carried out by the inventors, an acrylic device was designed having a channel width of about 3 mm, a channel length of about 5 cm, and a channel height of about 500 μm. The device had three layers held together by an adhesive and inlet ports and outlet ports were disposed in the top layer. The middle layer included three independent reaction wells and an independent channel associated with each well. Each channel has its own inlet and outlet disposed above in the top layer. The bottom layer was a solid piece of acrylic. Each reaction well comprised a matrix including PDMS comprising 4-methylumbelliferyl-β-D-glucoside.

The device was submerged in water contaminated with enterococci. A portion of the water entered each inlet and filled the channels and reaction wells. The device was then removed in a manner such that the channels and reaction wells remained saturated with the water. Each inlet and outlet port was sealed and the device was placed on a hotplate. Imaging with a CMOS camera of each well showed that there was no fluorescent signal when the device was initially placed on the hotplate and exposed to an array of excitation LEDs. The device was heated to about 41° C. for about 24 hours. Fluorescence increased over the course of the 24-hour period. At the end of the incubation period, imaging with a CMOS camera revealed a strong fluorescent signal coming from each well, which indicates that bacteria from enterococci reacted with the 4-methylumbelliferyl-β-D-glucoside, thereby producing a reaction product which emits a fluorescent signal. The fluorescent signal confirms the presence of enterococci and also confirms the utility of the present device.

All of the compositions and methods disclosed and claimed herein can be made and executed without undue experimentation in light of the present disclosure. While this invention may be embodied in many different forms, there are described in detail herein specific preferred embodiments of the invention. The present disclosure is an exemplification of the principles of the invention and is not intended to limit the invention to the particular embodiments illustrated. In addition, unless expressly stated to the contrary, use of the term “a” is intended to include “at least one” or “one or more.” For example, “a nutrient” is intended to include “at least one nutrient” or “one or more nutrients.”

Any ranges given either in absolute terms or in approximate terms are intended to encompass both, and any definitions used herein are intended to be clarifying and not limiting. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges (including all fractional and whole values) subsumed therein.

Any composition disclosed herein may comprise, consist of, or consist essentially of any element, component and/or ingredient disclosed herein or any combination of two or more of the elements, components or ingredients disclosed herein.

Any method disclosed herein may comprise, consist of, or consist essentially of any method step disclosed herein or any combination of two or more of the method steps disclosed herein.

The transitional phrase “comprising,” which is synonymous with “including,” “containing,” or “characterized by,” is inclusive or open-ended and does not exclude additional, un-recited elements, components, ingredients and/or method steps.

The transitional phrase “consisting of” excludes any element, component, ingredient, and/or method step not specified in the claim.

The transitional phrase “consisting essentially of” limits the scope of a claim to the specified elements, components, ingredients and/or steps, as well as those that do not materially affect the basic and novel characteristic(s) of the claimed invention.

As used herein, the term “about” refers to the cited value being within the errors arising from the standard deviation found in their respective testing measurements, and if those errors cannot be determined, then “about” may refer to, for example, within 5% of the cited value.

Furthermore, the invention encompasses any and all possible combinations of some or all of the various embodiments described herein. It should also be understood that various changes and modifications to the presently preferred embodiments described herein will be apparent to those skilled in the art. Such changes and modifications can be made without departing from the spirit and scope of the invention and without diminishing its intended advantages. It is therefore intended that such changes and modifications be covered by the appended claims.

1. A device, comprising:
a reaction well in communication with an external environment via a channel, the reaction well comprising a matrix, wherein the matrix comprises a reagent to carry out a reaction; and
an impermeable polymer that covers the reaction well;
wherein the channel comprises a first end and a second end, the first end comprising an inlet and the second end comprising an outlet.

2. The device of claim 1, wherein the reaction well is in fluid communication with the external environment.

3. The device of claim 1, wherein the reaction is a biological reaction, a chemical reaction, a biochemical reaction, or any combination thereof.

4. The device of claim 1, wherein the reagent comprises a nutrient and optionally an inhibitor.

5. The device of claim 4, wherein the nutrient comprises o-nitrophenyl-β-D-galactopyranoside (ONPG), 4-methylumbelliferyl-β-D-glucuronide (MUG), 4-methylumbelliferyl-β-D-glucoside, or any combination thereof.

6. (canceled)

7. The device of claim 1, wherein the device comprises a plurality of independent reaction wells.

8. The device of claim 7, further comprising a plurality of independent channels.

9. (canceled)

10. The device of claim 1, wherein the matrix comprises an elastomer.

11. The device of claim 1, wherein the matrix comprises polydimethylsiloxane.

12. The device of claim 1, wherein the impermeable polymer is thermoplastic, thermoset, machinable, manufacturable, transparent, translucent, biocompatible, chemically inert, resistant to ultraviolet light, or any combination thereof.

13. The device of claim 1, wherein the impermeable polymer comprises substantially no autofluorescence.

14. The device of claim 1, wherein the impermeable polymer comprises methyl methacrylate, polystyrene, polycarbonate, polypropylene, polyethylene, a cycloolefin polymer, and any combination thereof.

15. The device of claim 1, wherein the device comprises a first layer, a second layer disposed on the first layer, and a third layer disposed on the second layer, the second layer defining the channel and the reaction well, and the third layer defining the inlet and the outlet.

16. A method for water quality analysis, the method comprising:
immersing a device in an aqueous medium, wherein the device comprises a plurality of reaction wells and a transparent, impermeable polymer covering the plurality of reaction wells, wherein each reaction well comprises a matrix having a reagent to carry out a reaction and wherein each reaction well is in fluid communication with the aqueous medium via a channel;
heating the device at a predetermined temperature after immersing the device;
illuminating each of the plurality of reaction wells of the device with a fluorescence excitation source through the impermeable polymer;
detecting a fluorescence signal from each of the plurality of reaction wells with a corresponding fluorescence emission sensor through the impermeable polymer, wherein each fluorescence signal is indicative of whether fluorescent activity is detected in the corresponding reaction well; and
determining, by a computing device, a water quality metric based on the fluorescence signal from each of the plurality of reaction wells.

17. The method of claim 16, further comprising:
sealing the channel for each reaction well after immersing the device;
wherein heating the device further comprises heating the device after sealing the channel.

18. The method of claim 16, wherein determining the water quality metric comprises:
determining a number of the plurality of reaction wells for which the corresponding fluorescence signal is indicative of detected fluorescent activity; and
determining a most probable number (MPN) metric as a function of the number of the reaction wells for which the corresponding fluorescence signal is indicative of detected fluorescent activity.

19. The method of claim 18, wherein determining the number of the plurality of reaction wells for which the corresponding fluorescence signal is indicative of detected fluorescent activity comprises predicting the number with a machine learning model based on the fluorescence signal from each of the plurality of reaction wells.

20. The method of claim 19, wherein:
determining the water quality metric further comprises determining a derivative of the fluorescence signal from each of the plurality of reaction wells; and
predicting the number with the machine learning model further comprises predicting the number with the machine learning model based on the fluorescence signal and the derivative of the fluorescence signal from each of the plurality of reaction wells.

21. (canceled)

22. The method of claim 16, further comprising:
continuing to heat the device, illuminate the plurality of reaction wells, and detect the fluorescence signal from each of the plurality of reaction wells over a first time period between about 30 minutes to about 6 hours; and
determining the water quality metric based on the fluorescence signal from each of the plurality of reaction wells after expiration of the first time period.

23. The method of claim 16, wherein the aqueous medium comprises lake water, river water, or ocean water.

24. (canceled)

25. (canceled)
